(19) United States
(12) Patent Application Publication
(10) Pub. No.: US 2005/0234347 A1
(43) Pub. Date: Oct. 20, 2005

(54) Puncture-type endoscopic probe

(75) Inventors: Shuuichi Yamataka, Saitama (JP); Hiroshi Fujita, Saitama (JP)

Correspondence Address: SNIDER & ASSOCIATES, P. O. BOX 27613, WASHINGTON, DC, 20038-7613, US

(73) Assignee: Fujinon Corporation, Saitama-shi (JP)

(21) Appl. No.: 11/087,805

(22) Filed: Mar. 24, 2005

(30) Foreign Application Priority Data
Mar. 29, 2004 (JP) 2004-094727

(51) Int. Cl. A61B 8/00

(52) U.S. Cl. 600/476

(57) ABSTRACT

This puncture-type endoscopic probe of the invention comprises: an object optical system consisting of a fiber bundle which is composed of a plurality of optical fiber strands each having a light-emitting end which functions as a point light source, an imaging objective lens which focuses the light beams emitted from the light-emitting ends onto the suspected surface position and a light beam deflection member (prism) for deflecting light which is positioned between the light-emitting ends and the suspected surface position; and a puncturing section which is attached to the tip of the probe. The imaging objective lens is constituted by a micro lens array, which is positioned so that the light-emitting end positions and the suspected surface position are conjugated each other. The object optical system can be rotated around the rotational axis defined by the puncture direction.

### RELATED APPLICATIONS

This application claims the priority of Japanese Patent Application No. 2004-094727 filed on Mar. 29, 2004, which is incorporated herein by reference.

### BACKGROUND OF THE INVENTION

1. Field of the Invention

The present invention relates to a puncture-type endoscopic probe wherein the tip of the probe is inserted into the tissue in order to observe the same.

2. Description of the Prior Art

Conventionally, confocal scanning endoscopes have been used as endoscopic devices wherein detailed tomographic images of the lesion area are obtained for the detailed observation of the same (For example, refer to Japanese Unexamined Patent Publication No. 2000-121961).

Technology known as Optical Coherence Tomography (OCT) wherein tomographic images of the specimen are obtained using light interference is also being developed.

However, conventional confocal scanning endoscopes are only capable of obtaining optical slice images. In order to obtain tomographic images with an in-depth focus, an in-depth scan must be carried out while obtaining 2-dimensional data parallel to the surface of the specimen. The 3-dimensional data obtained must then be reconstructed to display the tomographic images. This requires a significant amount of time for data processing, where 3-dimensional tomographic images cannot be obtained instantaneously.

Thus, it remained difficult to obtain tomographic images with an in-depth focus together with endoscopic examinations, where rapid diagnoses are not possible.

Clear images could only be obtained within a range of approximately 100-150 μm, where it is difficult to observe lesions which are located below this area.

Furthermore, the method of measuring 3-dimensional tomographic images using the above-mentioned optical coherence tomography method had a lower resolution compared to the use of a confocal scanning endoscope. This occasionally resulted in unclear images.

### SUMMARY OF THE INVENTION

With the foregoing in view, it is an object of the present invention to provide a puncture-type endoscopic probe capable of carrying out both an accurate and suitable diagnosis based on rapidly-obtained, clear tomographic images with an in-depth focus together with endoscopic examinations.

In order to achieve the above object, the puncture-type endoscopic probe of the present invention is a puncture-type endoscopic probe which is inserted into the tissue using an endoscope in order to carry out the observation of the same, which comprises:

a fiber bundle composed of a plurality of optical fiber strands each having a light-emitting end which functions as a point light source;

an object optical system composed of imaging objective lenses for focusing the light beams emitted from the respective light-emitting ends onto the suspected surface position and a light beam deflection member for deflecting light beams which is positioned between the above light-emitting ends and the suspected surface position; and

a puncture section located at the tip of the probe.

Here, the above-mentioned imaging objective lenses should preferably be positioned so that the above light-emitting end positions and the above suspected surface positions are conjugated each other.

The above-mentioned imaging objective lenses should preferably be constituted by a micro lens array.

The above-mentioned object optical system should preferably be able to rotate around the rotational axis defined by the puncture direction.

The above mentioned light beam deflection member should preferably be constituted by a prism.

A protective section for containing the object optical system may be made of a cylindrical member and a translucent window section used for passing the light beams should preferably be attached at a circumferential direction to the member.

A sheath member for covering the puncturing section may be attached to the outside of the same and the sheath member may be moved along the probe in an axial direction in relation to the puncturing section.

### BRIEF DESCRIPTION OF THE DRAWINGS

FIG. 1 is a schematic sectional view showing the tip of the puncture-type endoscopic probe according to the embodiments of the present invention;

FIG. 2 is a schematic sectional view showing the rotation mechanism and the puncture-type endoscopic probe according to the embodiments of the present invention; and

FIG. 3 is a schematic diagram showing the implementation of inner tissue imaging using a puncture-type endoscopic probe according to the embodiments of the present invention.

### DESCRIPTION OF THE PREFERRED EMBODIMENTS

The embodiments of the puncture-type endoscopic probe of the present invention are described below with reference to the figures.

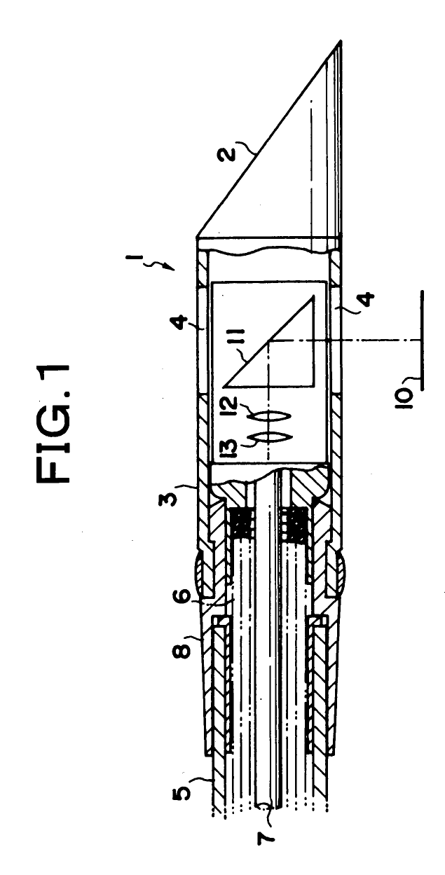

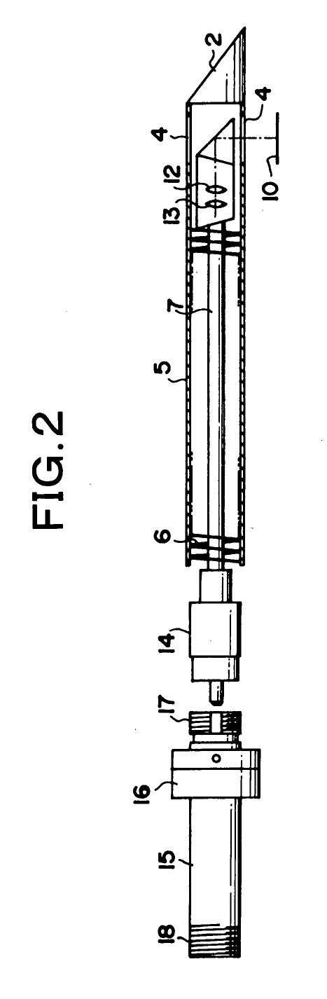

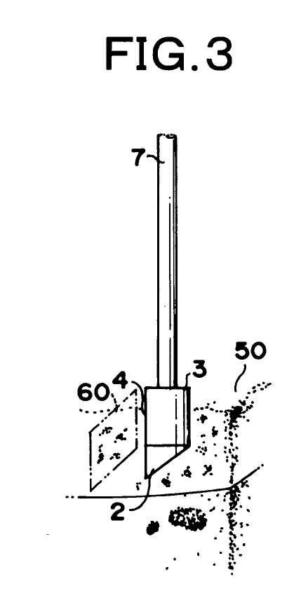

FIG. 1 and FIG. 2 are embodiments of the puncture-type endoscopic probe of the present invention. FIG. 1 is a schematic sectional view showing the tip of the probe and FIG. 2 is a schematic sectional view showing the probe and the rotation mechanism. FIG. 3 is a schematic diagram showing the implementation of inner tissue imaging using a puncture-type endoscopic probe related to the embodiments of the present invention.

In the puncture-type endoscopic probe 1, a protective section 3 which contains the object optical system which is attached to the tip of the flexible sheath 5 via the cylindrical connecting section 8 as shown in FIG. 1. A sectional roughly-triangular puncturing section 2 is attached at the tip of the protective section 3. The fiber bundle 7, whose outer circumference is sheathed in a helical spring 6, is contained within the sheath 5. The imaging objective lenses 12, 13 and the prism 11 which make up the object optical system are positioned at the tip of fiber bundle 7. Lens 13 is a collimator lens and lens 12 is an imaging lens. Lens 12 and lens 13 are jointly referred to below as imaging objective lenses 12, 13.

The fiber bundle 7 is composed of a plurality of optical fiber strands each having light-emitting end which functions as point light source. The imaging objective lenses 12, 13 are positioned so that the light-emitting end positions and the suspected surface position 10 are conjugated each other. In other words, the respective optical fiber strands hold the imaging objective lenses 12, 13 in place to make up the confocal optical system.

In order from the fiber bundle 7 side, the object optical system consists of the imaging objective lenses 12, 13 and the prism 11 as the optical path alteration member. The prism 11 forms a sectional right-angled isosceles triangle, the incline of which functions as a deflection surface that reflects light at a right angle. For example, of the imaging objective lenses 12, 13, the suspected surface position 10 of the inner issue can be shifted by moving the imaging lens 12 in the direction of the optical axis to obtain a wider-ranged tomogram.

The imaging objective lenses 12, 13 should preferably be made up of a micro lens array. In this case, 2-dimensional suspected surface data can be readily obtained by the use of a CCD as a light receiving device.

The protective section 3 containing the object optical system is made up of a cylindrical member which is equipped with a translucent window section 4 in a circumferential direction. The translucent window section 4 may be positioned continuously in a circumferential direction or may be divided into several windows in a circumferential direction.

The sheath 5, the connecting section 8, the protective section 3 and the puncturing section 2 of this puncture-type endoscopic probe 1 are all joined together as a single body. In order, this contains inside the helical spring 6, the fiber bundle 7 and the object optical system (imaging objective lenses 12, 13 and prism 11) which rotate as a single body. The diameter of the puncture-type endoscopic probe 1 is approximately 1-2 mm.

The plug 14 is located at the base end of the puncture-type endoscopic probe 1. A rotating operation section 15 can then be attached to the above plug 14.

The rotating operation section 15 is comprised of receptacles 17, 18 to either side and an operating ring 16 is attached to the outer circumference section. The receptacle 17 on the side of the tip is connected to the plug 14 located at the base end of the puncture-type endoscopic probe 1. The receptacle 18 on the side of the base is connected to the light source section and the imaging section (not shown in figure) via the extension section (not shown in figure).

The operating ring 16 is made to rotate together with the helical spring 6, fiber bundle 7 and the object optical system (imaging objective lenses 12, 13 and prism 11). Thus, by rotating the operating ring 16, the helical spring 6, fiber bundle 7 and the object optical system (imaging objective lenses 12, 13 and prism 11) can be rotated with the axial direction as the rotation axis.

Next, with reference to FIG. 3, the procedure for carrying out the imaging of the specimen is described using the puncture-type endoscopic probe 1 of the present embodiment.

In order to carry out the imaging of the specimen using the puncture-type endoscopic probe 1 of the present embodiment, the puncture-type endoscopic probe 1 must first be inserted in the vicinity of the specimen.

When the tip of the puncture-type endoscopic probe 1 reaches the vicinity of the surface of the tissue area 50 (such as lesion areas where the presence of cancer is suspected), the puncturing section 2 is thrust into the tissue area 50 and the translucent window section 4 is inserted into the tissue area 50. This enables 2-dimensional images of the suspected surface position 60 to be obtained when imaging is carried out in this way.

Also, rotating the operating ring 16 rotates the helical spring 6, fiber bundle 7 and the object optical system (imaging objective lenses 12, 13 and prism 11). This enables the interior of the tissue area 50 to be imaged in a circumferential direction wherein the puncture direction is the rotational axis direction.

The puncturing section 2 is then further inserted into the tissue area. Repeatedly carrying out the above-mentioned operation enables a wider-ranged, in-depth direction tomography of the interior of the tissue 50 to be obtained.

The tip section (puncturing section 2 and translucent window section 4) of the puncture-type endoscopic probe 1 is inserted into the tissue 50 at a depth of, for example, approximately 1-2 mm. The distance between the translucent window section 4 and the suspected surface position 60 is, for example, approximately 100-150 μm.

Although the sheath 5 of the present embodiment is taken to be flexible, a hard sheath may also be used. Similarly, light beam deflection members are not limited to prisms and may also consist of mirrors. Also, although the object optical system is made to rotate by manually operating the operating ring 16, the object optical system may also be made to rotate by using either a miniature motor or a combination of a manually-operated operating ring 16 and a miniature motor.

A photo diode can be used as a light receiving device by enabling suspected surface data to be obtained in time series by scanning incident light against the light fiber bundle.

Furthermore, by attaching a sheath member which covers the puncturing section 2 at the tip of the probe and enabling the above sheath member to move along the probe in an axial direction, the puncturing section 2 can be covered by the sheath member until the tip of the probe reaches the vicinity of the lesion area (such as the stomach wall). This prevents damage to other tissue due to the penetration of other tissue (such as the oral cavity and esophagus) by the puncturing section 2.

According to the puncture-type endoscopic probe of the present invention, the tip of the probe can be readily inserted into the tissue or specimen in order to obtain a satisfactory tomographic image at a specified depth.

The use of a fiber bundle composed of a plurality of optical fiber strands each having a light-emitting end which functions as a point light source (pinhole) enables the irradiation position of each light emitted from the respective optical fiber strands to be altered. This enables a 2-dimensional image to be obtained without the need for a scan to be carried out. Thus, there is no need for a complex mechanism, which allows the miniaturization of the probe as well as a reduction in the manufacturing cost of the same.

Furthermore, the use of a plurality of optical fiber strands which holds the objective lenses in place to make up the confocal optical system enables images with a high resolution to be obtained.

The use of micro array lenses as the imaging objective lenses enables both a compact optical system and a satisfactory image performance at the respective suspected surface positions. This enables both miniaturization of the probe and a reduction of manufacturing costs as well as an improvement in the imaging speed.

Furthermore, by using a rotatable object optical system wherein the puncture direction is the rotational axis direction, a single puncture enables circumferential imaging around the tissue at the puncture position. This minimizes damage caused to the tissue and yields a more detailed image over a wider range.

1. A puncture-type endoscopic probe which is inserted into the tissue using an endoscope in order to carry out the observation of the same, comprising:
a fiber bundle composed of a plurality of optical fiber strands each having a light-emitting end which functions as a point light source;
an object optical system composed of imaging objective lenses which is used to focus the light beams emitted from the respective light-emitting ends onto a suspected surface position and a light beam deflection member used to deflect light beams which is positioned between the light-emitting ends and the suspected surface position; and
a puncture section located at the tip of the probe.

2. The puncture-type endoscopic probe according to claim 1 wherein the imaging objective lenses are positioned so that the light-emitting end positions and the suspected surface position are conjugated each other.

3. The puncture-type endoscopic probe according to claim 1 wherein the imaging objective lenses are constituted by a micro lens array.

4. The puncture-type endoscopic probe according to claim 1 wherein the object optical system can be rotated around the rotational axis defined by the puncture direction.

5. The puncture-type endoscopic probe according to claim 1 wherein the light beam deflection member is constituted by a prism.

6. The puncture-type endoscopic probe according to claim 1 wherein a protective section for containing the object optical system is made of a cylindrical member and wherein a translucent window section used for passing the light beams is attached at a circumferential direction to the member.

7. The puncture-type endoscopic probe according to claim 1 wherein a sheath member which covers the puncturing section is attached to the outside of the same and wherein the sheath member is movable along the probe in an axial direction in relation to the puncturing section.
